(19) United States
(12) Patent Application Publication
(10) Pub. No.: US 2017/0003366 A1
(43) Pub. Date: Jan. 5, 2017

(54) SYSTEM AND METHOD FOR GENERATING MAGNETIC RESONANCE IMAGING (MRI) IMAGES USING STRUCTURES OF THE IMAGES

(71) Applicant: THE GENERAL HOSPITAL CORPORATION, Boston, MA (US)

(72) Inventor: Kourosh Jafari-Lhouzani, Boston, MA (US)

(21) Appl. No.: 15/113,327

(22) PCT Filed: Jan. 23, 2015
(86) PCT No.: PCT/US15/12594

§ 371 Date: Jul. 21, 2016

Related U.S. Application Data
(60) Provisional application No. 61/930,716, filed on Jan. 23, 2014.

(51) Int. Cl. G01R 33/56; G06T 7/00; A61B 5/00; A61B 5/0402; A61B 5/08; G06T 11/00; G01R 33/567

(52) U.S. Cl. CPC G01R 33/5608; G06T 11/003; G06T 7/0012; G06T 7/0085; G01R 33/5673; G01R 33/5601; A61B 5/0402; A61B 5/08; A61B 5/7285; G06T 2207/10088; G06T 2207/30016

(57) ABSTRACT

A system and method for generating high resolution (HR) images from low resolution (LR) images or data by selectively choosing neighbors and the tissue types of the neighbors when estimating the image intensity of a voxel with the values of the neighbors. The system and method may interpolate LR images of a first contrast with the help of the high resolution HR images of a second contrast using the anatomical structures in both sets of images.

### CROSS-REFERENCE TO RELATED APPLICATIONS

This application claims the benefit of, and herein incorporates by reference in its entirety, U.S. Provisional Patent Application Ser. No. 61/930,716, filed on Jan. 23, 2014, and entitled “Upsampling MRI Images.”

### STATEMENT REGARDING FEDERALLY SPONSORED RESEARCH

This invention was made with government support under R90-DA023427, 5U01CA154601-03, 2P41EB015896-16, R01CA129371, and K24CA125440A awarded by the National Institutes of Health. The government has certain rights in the invention.

### BACKGROUND OF THE INVENTION

In quantitative analysis of medical images, certain spatial resolution is desired. In magnetic resonance imaging (MRI), the acquisition time, short physiological phenomena, and organ motion may limit the image resolution. For example, in dynamic contrast enhanced (DCE) and dynamic susceptibility contrast (DSC) MRI of brain, a tracer is injected into the vein during continuous rapid brain imaging. As the arrival of the bolus, its passage through the brain, and wash-out occur in a short period of time, rapid imaging is required to achieve sufficient temporal resolution to capture the dynamics of the contrast. This is often achieved by sacrificing the spatial resolution. In some cases, spatial resolution is limited by image acquisition time. For example, fluid attenuated inversion recovery (FLAIR) MRI requires longer acquisition time than T₁-weighted (T_{1W}) MRI. Thus, spatial resolution may be compromised to achieve reasonable scan time and minimize the likelihood of motion artifact. This is usually done by acquiring relatively small number of two dimensional (2-D) slices resulting in large slice thickness and spacing between slices. As a result, the images have lower resolution in the slice dimension than the other two dimensions (acquisition plane), resulting in highly anisotropic voxels (e.g., 1 mm×1 mm×6 mm). The images may alternatively be undersampled in the k-space of MRI to save time. This is called Compressed Sensing (CS). The images are then usually reconstructed using reconstruction algorithms based on sparsity constraints. The quality of reconstruction, however, is limited resulting in poor quality MR images.

Quantitative analyses of these low resolution (LR) images are further distorted when these images are transformed into a different space. For instance, voxel-wise analysis usually requires all image volumes of one subject to be aligned and analyzed in one space. Thus, if slices of different image volumes are not already acquired exactly from the same location, they need to be coregistered. Coregistration, in this case, involves guessing or interpolation. Unfortunately, guessing or interpolation techniques often inject further errors into the process, which can degrade the quality and value of the images clinically.

Therefore, it would be desirable to have a method for generating high resolution MRI images with high image quality, despite a lack of data for high resolution MRI images being unavailable.

### SUMMARY OF THE INVENTION

The present invention overcomes the aforementioned drawbacks by providing a system and method to interpolate low resolution (LR) images of a first contrast with the help of high resolution (HR) images of a second contrast using anatomical structures in both sets of images. To preserve the contrast in the LR images of the first contrast but prevent the dominance of the errors in them due to their low resolution, the weights used to express the interpolated images can, optionally, be calculated more than once. In the first time, the LR images may be interpolated to the high resolution and HR images of the first contrast are generated. The two sets of HR images are then coregistered. Weights are calculated using the laminar structures of the coregistered HR images of the second contrast, and then are used to generate HR images of the first contrast with less errors. In the second time, weights are calculated again using the laminar structures of both the coregistered HR images of the second contrast and the generated HR images of the first contrast with less errors, and then may be used to generated final interpolated images. In one configuration, the laminar structures are represented with feature vectors comprising image intensities, edges, and propagated edge information and weights are calculated based on the probabilities that neighbors of a given voxel have similar feature vectors to the given voxel.

In accordance with one aspect of the disclosure, a method is provided for generating magnetic resonance imaging (MRI) images of a subject. The method includes acquiring low resolution (LR) MRI images of a first contrast and high resolution (HR) images of a second contrast, generating HR images of the first contrast by interpolating the LR images of the first contrast to desired resolutions with a first interpolation method, and c coregistering the HR images of the second contrast with the HR MRI images of the first contrast to generate coregistered HR images of the second contrast and coregistered HR images of the first contrast. The method also includes estimating first weights using laminar structures of the coregistered HR images of the second contrast and generating first interpolated HR images of the first contrast using the first weights and the coregistered HR images of the first contrast. The method further includes estimating second weights based on the first interpolated HR images of the first contrast and the coregistered HR images of the second contrast using laminar structures of the first interpolated HR images of the first contrast and the laminar structures of the coregistered HR images of the second contrast and generating final interpolated HR images of the first contrast using the second weights and the first interpolated HR images of the first contrast.

In accordance with another aspect of the disclosure, a method is provided for generating weights used in MRI image interpolation to create desired images. The method includes acquiring MRI images, detecting edges of the MRI images, and propagating edge information of the MRI images by filtering the MRI images with blurring filters and therein generating blurred images. The method also includes generating feature vectors of the MRI images, wherein the vectors include image intensities of the MRI images, the edges, and the blurred images and generating probability for each voxel of the MRI images that neighbors of the each voxel having the feature vectors similar to the each voxel. The method further includes generating a weight for the each voxel based on the probability for the each voxel, wherein image intensity at a given voxel of interpolated images of the MRI images is represented as a sum over neighbors of the given voxel of the weight of each of the neighbors multiplied by image intensity of the each of the neighbors.

In accordance with another aspect of the disclosure, a magnetic resonance imaging (MRI) system is provided that includes a magnet system configured to generate a polarizing magnetic field about at least a portion of a subject arranged in the MRI system and a magnetic gradient system including a plurality of magnetic gradient coils configured to apply at least one magnetic gradient field to the polarizing magnetic field. The MRI system also includes a radio frequency (RF) system configured to apply an RF field to the subject and to receive magnetic resonance signals from the subject using a coil array and a computer system. The computer is programmed to acquire MRI images acquired using the MRI system, detect edges in the MRI images to compile edge information, and propagate edge information of the MRI images by filtering the MRI images with filters to generate filtered images. The computer is further programmed to generate feature vectors of the MRI images, wherein the vectors are generated using image intensities of the MRI images, the edge information, and the filtered images and generate a probability for each voxel of the MRI images that has a neighbor having the feature vectors similar to the each voxel. The computer is further programmed to generate a weight for the each voxel based on the probability for the each voxel. The image intensity at a given voxel of interpolated images of the MRI images is represented as a sum over neighbors of the given voxel of the weight of each of the neighbors multiplied by image intensity of the each of the neighbors.

The foregoing and other aspects and advantages of the invention will appear from the following description. In the description, reference is made to the accompanying drawings which form a part hereof, and in which there is shown by way of illustration at least one embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an MRI system for use with the present invention.

FIG. 2 is a flowchart setting forth example steps of a method for generating MRI images implemented in accordance with the present application.

FIG. 3 is a flowchart setting forth example steps of a method for generating weights used in interpolating MRI images implemented in accordance with the present application.

FIG. 4 is a picture of cortex depicting its laminar structure.

FIG. 5 (a) is an original image with a point of interest marked by a cross sign.

FIG. 5(b) is a similarity map showing image pixels having similar tissue type to the point of interest in FIG. 5(a) using a 3×3 patch.

FIG. 5(c) is similarity map taking into account the laminar structure of MR images.

FIG. 6(a) is an image with laminar structure.

FIG. 6(b) shows contour lines depicting of edges of the image using a gradient operator (before applying the gradient operator, the image is slightly smoothed).

FIG. 6(c) shows a filtered image of the image in FIG. 6(b) with Gaussian filters of different standard deviations.

FIG. 6(d) shows a filtered image of the image in FIG. 6(b) with larger standard deviation than used in FIG. 6(c).

FIG. 7(a) is a graph showing experimental results where performance is achieved with Nᵥ=10, but PSNR is reduced at most by 0.2 when other values of Nᵥ are used.

FIG. 7(b) is a graph showing the effect of neighborhood size on PSNR.

FIG. 7(c) is a graph showing the effect of the number of Gaussian kernels and their standard deviations.

FIG. 7(d) is a graph showing the number of Gaussian kernels change the PSNR but, similar to standard deviation of the kernels.

FIG. 7(e) is a graph showing PSNR curves with two weight updates.

FIG. 7(f) is a graph showing the effects of slice thicknesses on PSNR.

### DETAILED DESCRIPTION OF THE INVENTION

As discussed, quantitative analyses of these low resolution (LR) images are often distorted when images are transformed into a different space. For instance, voxel-wise analysis usually requires all image volumes of one subject to be aligned and analyzed in one space. Thus, if slices of different image volumes are not already acquired exactly from the same location, they need to be coregistered. Coregistration, in this case, involves guessing or interpolation. However, standard interpolation techniques are not accurate and result in distorted edges in the planes perpendicular to the acquisition plane. The reason for an inaccurate interpolation is that the neighboring voxels, from which the intensity of an unknown voxel is estimated, may not have the same tissue type as the unknown voxel. This results in an incorrect estimation of the voxel intensity using standard interpolation techniques.

As will be described, the present disclosure provides a system and method for generating high resolution images from low resolution images or data by selectively choosing neighbors and the tissue types of the neighbors when estimating the image intensity of a voxel with the values of the neighbors. Thus, a system and method is provided to interpolate low resolution (LR) images of a first contrast with the help of high resolution (HR) images of a second contrast using the laminar structures in both sets of images. To preserve the contrast in the LR images of the first contrast but prevent the dominance of the errors in them due to their low resolution, the weights used to express the interpolated images can be calculated more than once.

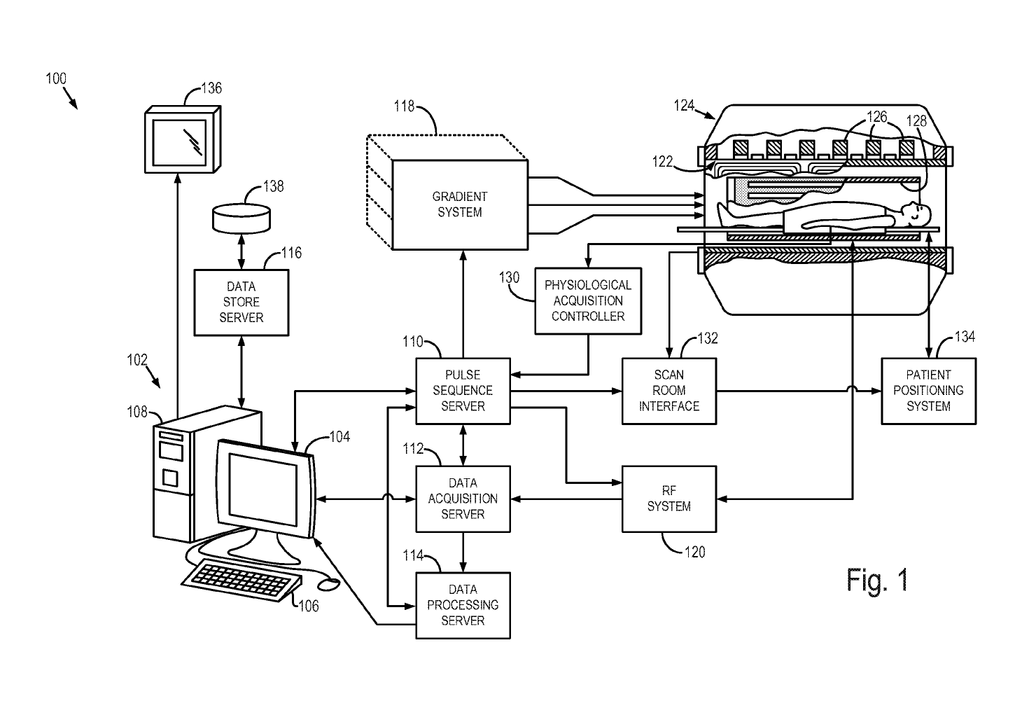

Referring particularly now to FIG. 1, an exemplary magnetic resonance imaging (“MRI”) system 100 is illustrated. The following discussion will be made with reference to MRI data and MRI images. However, other imaging modalities are contemplated, including computed tomography, ultrasound, and the like. The MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114; and a data store server 116. The workstation 102 and each server 110, 112, 114, and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128.

RF excitation waveforms are applied to the RF coil 128, or a separate local coil (not shown in FIG. 1), by the RF system 120 to perform the prescribed magnetic resonance pulse sequence. Responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays (not shown in FIG. 1).

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} (1);

and the phase of the received MR signal may also be determined:

$$ϕ = {tan}^{- 1} ( \frac{Q}{I} ) . \qquad ( 2 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

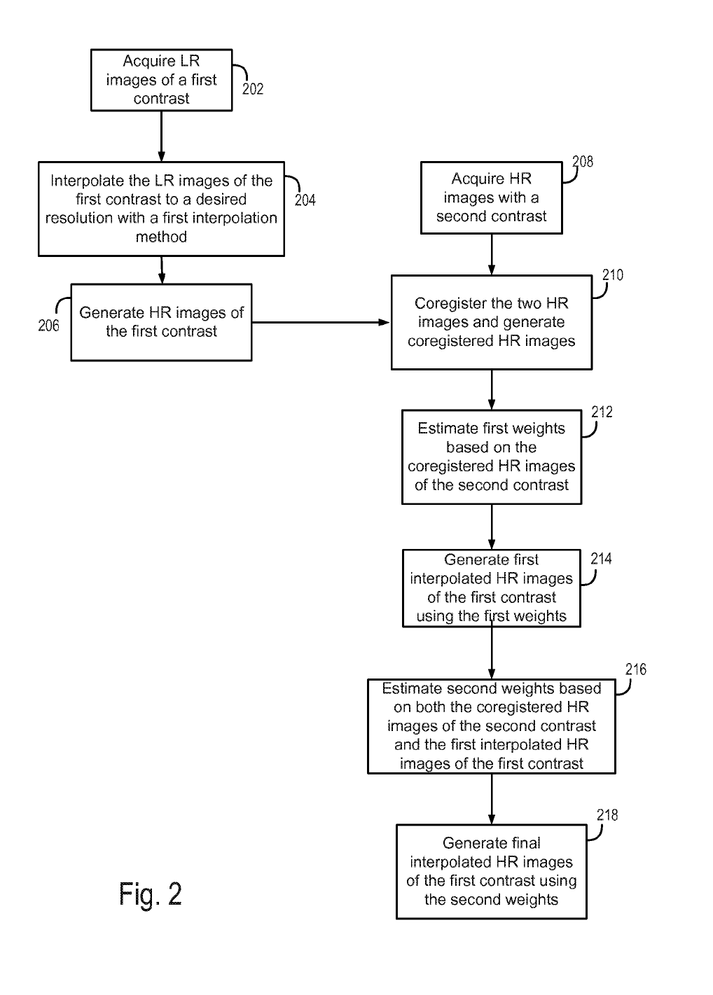

As shown in FIG. 1, the radiofrequency (“RF”) system 120 may be connected to the whole body RF coil 128, or, as shown in FIG. 2, a transmission channel 202 of the RF system 120 may connect to a RF transmission coil 204 and a receiver channel 206 may connect to a separate RF receiver coil 208. Often, the transmission channel 202 is connected to the whole body RF coil 128 and each receiver section is connected to a separate local RF coil.

The above-described system can be used to acquire different types of data, including so-called high resolution (HR) MRI images and low resolution (LR) MRI images. As used herein, “HR” and “LR” are relative and “LR images” refers to images having a resolution less than that of “HR images.” As one non-limiting example, HR images may have a resolution of approximately 1 mm×1 mm×1 mm or higher resolution and LR images may have a resolution of less than that of the HR images, such as 1 mm×1 mm×6 mm or 3 mm×3 mm×3 mm. As will be described, the systems and methods of the present invention can be used to generate HR images of LR images. The present invention provides final images that surpass traditional processes for providing “enhanced” or higher resolution images.

For example, one such traditional processes for “enhancing” or providing higher resolution images is interpolation. In interpolation, the value of a voxel is estimated as a function of the values of its neighboring points. For example, in linear interpolation, the intensities are assumed to be a linear combination of the values of neighboring points. Other methods have been proposed to improve the interpolation of lower resolution images. In one category of these methods, a single lower resolution image acquired from the subject is available for “upsampling.” Adjacent slices of the lower resolution image may be registered using a non-rigid method to reconstruct the slices between the slices. These registration-based methods work best when each slice is acquired from a thin slab of the subject and blurred edges between thick slices lead to poor coregistration. In one method, a family of adaptive three dimensional (3-D) interpolation filters are designed and conditioned on different spatial contexts in order to reconstruct a slice based on four neighboring slices. This method requires a training set. In another method, a sparse super-resolution approach was proposed using overcomplete dictionaries. This method also requires a training set.

The method disclosed in the present application uses an HR image with a different contrast to upsample an LR image. The HR image may be a T_{1W} image with isotropic voxel size that is acquired to reveal the brain structure. An LR image may be interpolated with the help of this HR image.

Referring now to FIG. 2, an example method for generating MRI images implemented in accordance with the present application is provided. In step 202, LR MRI images of a first contrast are acquired. In step 208, HR MRI images of a second contrast are acquired. The images may be retrieved from an MRI system, storage server associated with the MRI system, or other storage or memory that is separate or remote from the MRI system. Such a device may be a computer, a workstation, or a mobile device. In step 204, the LR images of the first contrast are interpolated to a desired resolution with a first interpolation method. Such an interpolation method may include a variety of interpolation methods, such as nearest neighbor interpolation method or linear interpolation method. The desired resolution that images are interpolated into is often higher than that of the LR images. So with step 204, HR images of the first contrast are generated in step 206. In step 210, the two HR images are co-registered to generate co-registered HR images of the first or second contrast. In step 212, first weights are estimated taking into account the structures of the co-registered HR images of the second contrast. As will be described, the structure, in some clinical applications, may be the laminar structure. In step 214, using the first weights, first interpolated HR images of the first contrast are generated. In step 216, second weights are estimated taking into account the structures of both the co-registered HR images of the second contrast and the first interpolated HR images of the first contrast. In step 218, the final interpolated HR images of the first contrast are generated.

Particularly, in one configuration, the intensity of a voxel is approximated by the weighted linear combination of all intensities within a neighborhood of the voxel as in Equation (3):

$$x ( v ) \approx {\sum }_{k ∈ Ω ( v )} w ( v , k ) x ( k ) ; \qquad ( 3 )$$

where x is the image intensity function and ω(v) is a neighborhood of voxel v. The weights w(v, k) can be calculated based on the similarity of 3-D neighborhoods Ω centered in voxels v and k in the HR image. The same weights may be used for interpolating the LR image based on the assumption that the local patterns in HR and LR images are similar. This assumption may not be valid, for example, when a lesion is clearly visible in the LR image but not in the HR image due to their different contrasts. To address this issue, weights can be calculated based on both the HR and the LR images.

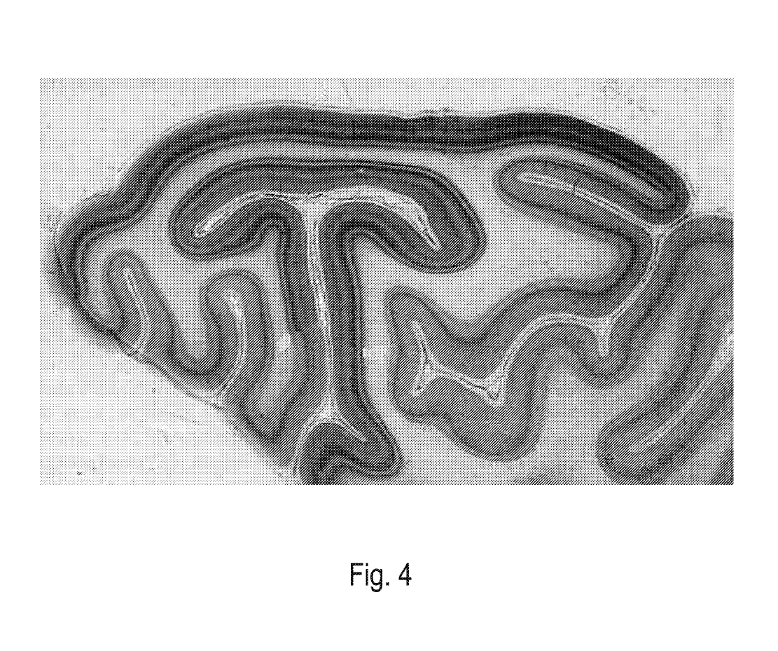

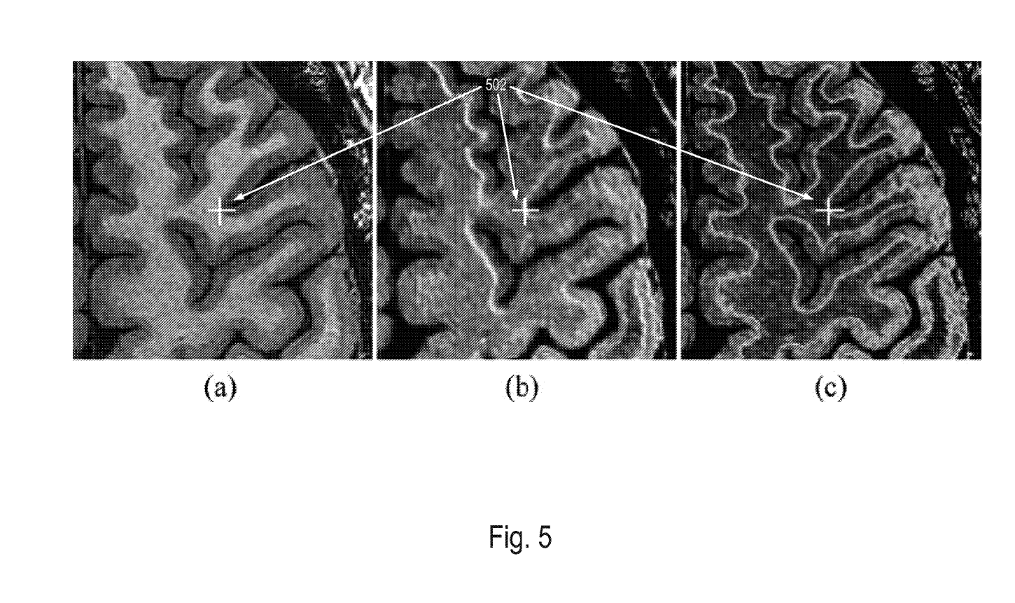

The weights w(v, k) in the combination of neighboring voxels to generate the image intensity at voxel v in Eqn. (3) may be proportional to the similarity between the image intensity x(v) at voxel v and the image intensity x(k) at neighboring voxel k. But conventionally, w(v, k) correlates with the distance between points v and k, based on the assumption that points closer to each other are more likely to have similar values. This assumption does not always stand, particularly, in images with highly anisotropic voxels as tissue of different structure features may be close to each other while tissue of similar structure features may be in a long layer. For example, as will be described, this situation is illustrated in FIGS. 4 and 5(c).

In addition, the chosen neighborhoods should suit the structural features of the images to be interpolated. Referring to FIG. 4, a picture depicting the laminar structure of brain cortex is provided. As shown, the cortex is made of layers of cells that form a laminar structure. Voxels in a layer have a similar tissue type.

As mentioned above, the structure considered in steps 212 and 214 of FIG. 2 may be laminar structure. To take into account the laminar structure, voxels that have equal distances to a strong edge may be assumed to be likely to belong to the same tissue type. In other methods, neighborhoods may be assumed to be patches, which are rotation variant and better suit images with directional textural patterns. Such an assumption on neighborhoods does not suit MR images. As a result, two close voxels of one tissue type placed at curved structures may be interpreted as dissimilar because they may be interpreted as belonging to different patches. Referring to FIG. 5, the effects of different approaches on generating similarity maps are provided. Higher values (brighter points) show higher similarity. The point of interest 502 is located at the boundary of white matter and gray matter. FIGS. 5(a) is the original image with a point of interest 502 marked by a cross sign, (b) is a similarity map showing image pixels having similar tissue type to the point of interest 502 using a 3×3 patch, (c) is similarity map taking into account the laminar structure of MR images. As shown in FIG. 5(b), the laminar shape of the brain structure is ignored by a patch-based approach and many voxels that may be in the same gyrus or sulcus are not depicted as similar to voxel 502. To the contrary, as shown in FIG. 5(c), if the laminar structure of MRI images is taken into account, the similarity map shows that voxels with similar structure features with that of voxel 502 align with the laminar structure. This assumption of laminar structures is not only valid for normal brain tissue, but also for lesions, such as tumors and lesions associated with multiple sclerosis, epilepsy, and schizophrenia. As one of skill will appreciate, the above discussion of laminar structure and strategies for creating improved images in the face of such structural components in images can be extended to a variety of other anatomical structures. As one of skill will be appreciated, anatomy include a variety of structures that, when imaged using MRI or other imaging modalities, yields images having boundaries or edges such as described above, including those with curves.

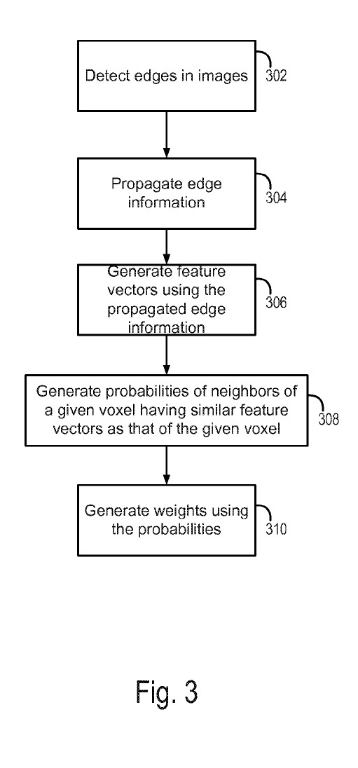

Referring now to FIG. 3, a flowchart setting forth an example of steps for a method for generating weights used in interpolation is provided. In step 302, edges in the images are detected. The edge information is propagated in step 304 where edge information is propagated in a direction perpendicular to the edges to voxels in layers parallel to the edges so to capture the layered structures in the images. Using the propagated edge information, feature vectors are generated in step 306. In step 308, probabilities of neighbors having similar feature vectors are generated for each voxel. In step 310, weights are generated using the probabilities.

Particularly, in one configuration, two points are considered more similar if they have similar neighborhoods. One way to capture this relation is expressed as:

$${w}_{x} ( v , k ) = \frac{1}{N} P ( v , k ) ; \qquad ( 4 )$$

where P(v, k) is the probability that voxels v and k have similar tissue types in terms of tissue properties specified by intensity function x, and N is a normalization factor such that Σₖw(v, k)=1. Equation (4) means that the image intensity of a voxel should be interpolated using neighboring voxels that are similar in tissue property to that voxel. The function P(v, k) may be estimated from a HR image of the same subject. If the contrast of the two images is different, they may have different values of wₓ(v, k). But if the two images have some structural similarity, it is likely that the above estimation results in more accurate interpolation than other methods.

In one configuration, the probability of neighbors having similar tissue type is expressed as:

P(v, k)∝ exp(−∥F(v)−F(k)∥²) (5);

where F is a feature vector representing tissue property of a voxel. Assuming that z represents the intensity of the HR image with a different contrast coregistered to the LR image, F could be set to z or a vector of z values in a neighborhood of the input voxel, e.g., a patch. But such a feature vector does not take the brain laminar structure into account and is not rotation-invariant.

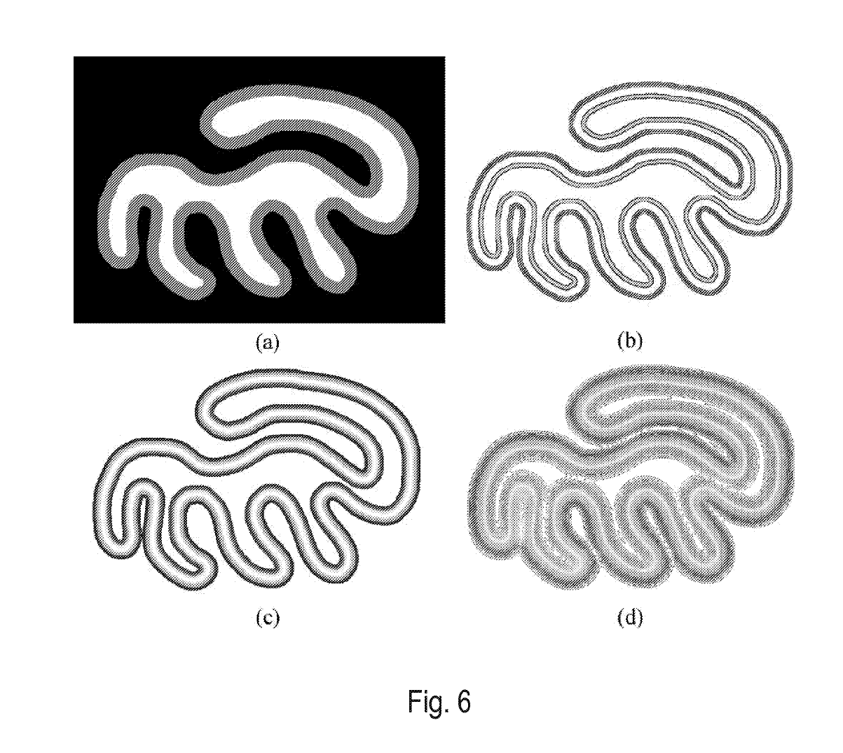

To capture the laminar or similar structures, voxels having equal distances to the main edges may have similar values. Equivalently, contour maps may have isolines parallel to the main edges. The edge information may be propagated in a direction perpendicular to the edge direction. A gradient operator can be used to extract edges. Referring to FIGS. 6(a)-(d), an image with laminar structure and the effects of propagating its edge information are depicted. In FIG. 6(a), an image with laminar structure is provided. FIG. 6(b) shows contour lines depicting of edges of the image using a gradient operator (before applying the gradient operator, the image is slightly smoothed) FIGS. 6(c) and 6(d) show filtered images of the image in (b) with Gaussian filters of different standard deviations, where a larger standard deviation is used in FIG. 6(d) than FIG. 6(c). Note in FIGS. 6(c) and 6(d) that the edge information stays local in a gradient operator and is also propagated further from the edges with the filtering. Edges and propagated edge information can closely characterize a laminar structure.

To propagate edge information, a multi-resolution blurring filtering approach is used. In one configuration, Gaussian filters are used and the image is filtered by a set of Gaussian kernels with different standard deviations. Referring to FIGS. 6(c) and 6(d) again, FIGS. 6(c) and 6(d) show the contour maps of the filtered image using Gaussian kernels with two different standard deviations. By increasing the standard deviation (as in FIG. 6(d)), the edge information is propagated further, but, in the mean time, detailed information of smaller structures is lost. To propagate edge information and also preserve detailed information, the gradient operator and Gaussian filters may be combined into the similarity vector to retain both high and low resolution information. That is, the following feature vector can be used:

F_{z}(v)=(z(v), |∀z(v)|, z*g₁|ᵥ, z*g₂|ᵥ, . . . , z*gₖ|ᵥ)^{T} (6);

where ∀ is gradient operator, * is convolution operator, g₁, i=1, 2, . . . , k are Gaussian kernels, and k is the number of kernels. The required standard deviations of the Gaussian kernels depend on the desired voxel size for the upsampled image. Referring again to FIG. 5, the method disclosed in the present application perform superior in finding voxels with similar tissue types in a laminar structure (shown in FIG. 5(c)). Similarity map is calculated faster with the method disclosed herein than a patch-based method if the vector size is small because fewer mathematical operations would be required.

In interpolating an LR image, one issue needs to be considered: an LR image may not be just a down-sampled version of a higher resolution image. Partial volume effect and geometric transformation are usually present in the LR images. A degradation model that takes those into account sums up the relation between an LR image and its original HR image before degrading:

y=Hx+n (7);

where y is the LR image, x is the original HR image of the same contrast before the degrading, H is a matrix incorporating geometric transformation, partial volume effect, and sub-sampling, and n is the observation noise. The HR image can be estimated from the LR image based on the degradation model using estimation methods. For example, one way to estimate the HR image is to minimize a least-square cost function such as

$${x}^{⋒} = {arg min}_{x} \vert \vert y - Hx {\vert \vert}^{2} . \qquad ( 8 )$$

As this problem does not have a unique solution, some prior information is needed to constrain the solutions. The prior information may be applied in the form of Eqn. (3), for example by introducing a regularization term and solving:

$${x}^{⋒} = {arg min}_{y} \vert \vert y - Hx {\vert \vert}^{2} + λ R ( x ) ; \qquad ( 9 )$$

where R(x) incorporates the constraint in Eqn. (3) as:

$$R ( x ) = {\sum }_{v} \vert x ( v ) - {\sum }_{k ∈ Ω ( v )} w ( v , k ) x ( k ) {\vert}^{2} . \qquad ( 10 )$$

His assumed known. So the next step is to develop a minimization algorithm.

In one configuration, an iterative method is used for the minimization in Eqn. (9). In iteration step t+1, the image \hat{x}_{rec}^{t+1} is reconstructed by Eqn. (3) from \hat{x}_{rec}^{t} with the condition in Eqn. (5) enforced as:

\hat{x}^{t+1}=\hat{x}_{rec}^{t+1}−NN(H\hat{x}_{rec}^{t+1}−y) (11);

where NN is the nearest neighbor interpolation operator. As noted above, other conventional interpolation methods, such as linear interpolation, can be used in the place of nearest neighbor interpolation method. To take into account the inconsistencies between the LR and HR weights, the values of wₓ(v, k) are generated using both HR and LR images as follows:

$${w}_{x} ( v , k ) = \frac{1}{N} exp ( - {α}_{z} \vert \vert {F}_{z} ( v ) - {F}_{z} ( k ) {\vert \vert}^{2} - {α}_{x} \vert \vert {F}_{x} ( v ) - {F}_{x} ( k ) {\vert \vert}^{2} ) ; \qquad ( 12 )$$

where α_{z} and αₓ denote the contribution of the features of LR and HR images. Consider a situation in which a lesion is visible in the LR image but not in the HR image. Such an example may occur when F_{z} values are similar in a neighborhood, but where Fₓ values are not. In this case, the LR image should be dominant in Eqn. (12).

Besides image quality, speed and accuracy need to be considered in generating the wₓ(v, k) values. First is speed. The most time-consuming part of the method can be generating the wₓ(v, k) values. The algorithm can become slowed if the weights are recalculated in each iteration. Second is accuracy. Taking the LR image into account during each iteration in the calculation of weights may result in suboptimal solutions because the structural information of the LR image—which may not be accurate in the first place—may gradually dominate and reduce the accuracy as iterations proceed. To expedite the process and avoid undesired interpolation, the weights may be first calculated using only the HR image (i.e., by setting αₓ=0 in Eqn. (12)). The LR image can then be upsampled using these weights in an iterative approach for the minimization in Eqn. (9). This iteration process is relatively fast. The upsampled LR image then becomes more similar to the correct result and thus can better contribute to the upsampling. In the next step, a nonzero αₓ is used to calculate weights wₓ(v, k) and the image is upsampled iteratively with the new weights. This way the weights are only calculated twice in total. In one configuration, to save memory and time, the highest Nᵥ of wₓ(v, k) values are kept in the calculation assuming that there are Nᵥ voxels with similar properties to the voxel of interest, and that the rest of voxels can be ignored. Nᵥ can be adjusted based on the neighborhood size and voxel size.

An example algorithm is summarized as below:

1) Use NN operator to generate initial estimation \hat{x}⁰.

2) Coregister the HR image of the second contrast to \hat{x}⁰ and call the coregistered HR image of the second contrast z.

3) Set αₓ=0.

4) Estimate the weighs wₓ(v, k) using z and \hat{x}⁰.

5) Start with t=0.

6) Repeat the following steps a)-c) until ∥\hat{x}^{t+1}−\hat{x}^{t}∥²<ε:
a) Reconstruct \hat{x}^{t+1} from \hat{x}^{t} using Eqn. (3);
b) Correct \hat{x}^{t+1} for degradation by applying Eqn. (11);
c) Increment t by 1.

7) Repeat steps 4-6 with a nonzero αₓ.

In the example algorithm, first a NN operator interpolates the image to generate an initial estimation of the upsampled image. Slices can be inserted to make the voxels of the initial upsampled image near-isotropic. If a HR image of a different contrast is used, the HR image is then coregistered to the initial upsampled image so that each corresponding voxel in the HR and the initial upsampled images represents the same location in the subject. Afterwards, the values of wₓ(v, k) are estimated for each voxel using the coregistered HR image. Using these weights, a mid-step upsampled image is generated with an iterative method. Afterward, the value of wₓ(v, k) are estimated again, this time with a nonzero αₓ. This means that both the coregistered HR image and the mid-step image are used to calculate the weights to take into account of the inconsistencies of the contrasts between the LR and the HR images. Afterwards, a final up sampled image is generated with the new weights and the same iterative method used in generating the mid-step upsampled image.

To evaluate the effects of the number of voxels, neighborhood size, number of Gaussian kernels and their standard deviations, parameter ε, and slice thickness on the quality of interpolation using the method disclosed herein, peak signal-to-noise ratio (PSNR) is used. PSNR is defined as

$$PSNR ( \hat{x} ) = 10 {log}_{10} ( \frac{{d}^{2}}{MSE ( \hat{x} )} ) ; \qquad ( 13 )$$

where \hat{x} is the reconstructed image, MSE(\hat{x})=1/|Ω|. Σ_{VεΩ}(x(v)−\hat{x}(v))², Ω is the region covering the subject depicted in the image, i.e., the whole image excluding background, and d is the actual dynamic range of the image, i.e., d=max(x)−min(x).

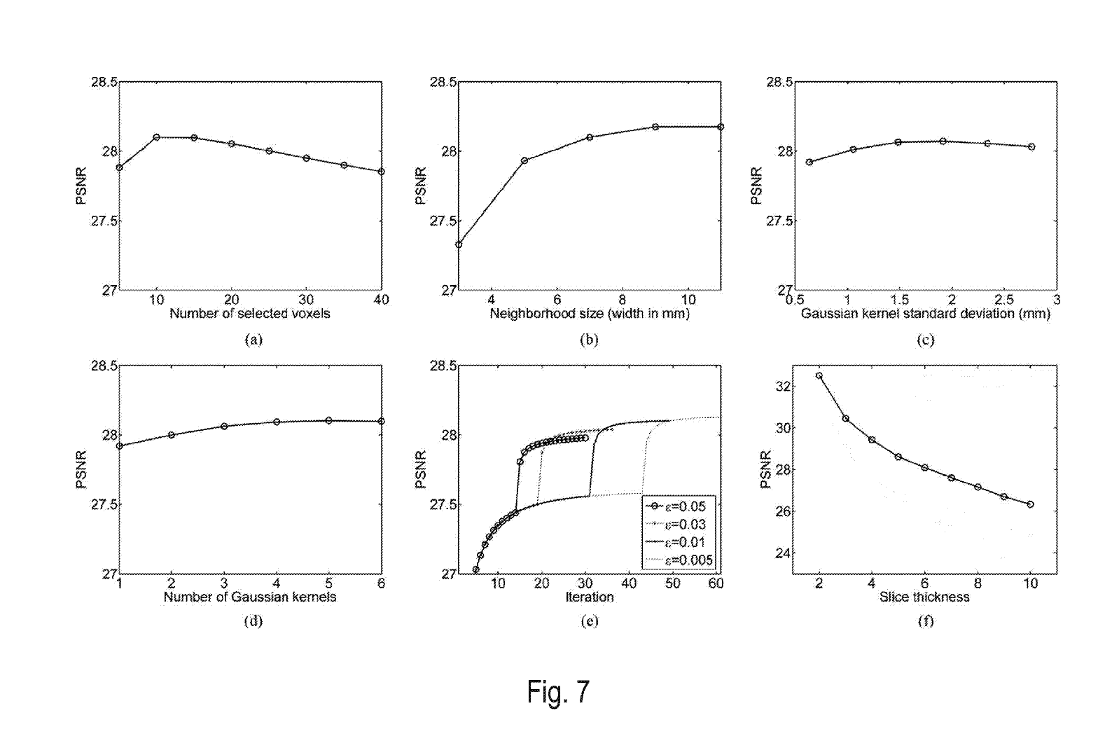

Referring now to FIGS. 7(a) through 7(f), the results of the evaluation are presented. Choosing a small number of selected voxels, Nᵥ, makes the method faster and requires less memory to store the weight array. As shown in FIG. 7(a), the best performance is achieved with Nᵥ=10, but PSNR is reduced at most by 0.2 when other values of Nᵥ are used. The optimal value of Nᵥ may, however, change with the desired resolution for the upsampled image. The use of only 10 voxels for reconstruction also makes the method less memory- and calculation-demanding.

The effect of neighborhood size on PSNR is shown in FIG. 7(b) for Nᵥ=10. As shown, as the neighborhood size increases, computation time also increases but the increase in the PSNR saturates. In one experiment, a neighborhood size of 7 mm (i.e., a cube of size 7×7×7 around the voxel of interest in the image with 1 mm×1 mm×1 mm resolution) is chosen to balance the computation time and PSNR. Similar to Nᵥ, the optimal or desired neighborhood size may change with the desired resolution for the upsampled image.

To evaluate the effect of the number of Gaussian kernels and their standard deviations, six filters with standard deviations of σₙ=(2n+1)h and filter size (2n+1)×(2n+1)×(2n+1), where n=1, 2, . . . , 6 and h=1/(4\sqrt{2ln2}) are used. In one experiment, single Gaussian kernels (k=1) with standard deviations σₙ, n=1, 2, . . . , 6 are used to evaluate the effect of standard deviation on the upsampling accuracy. The results are presented in FIG. 7(c). As shown, PSNR changes with the standard deviation. But the change in PSNR was less than 0.2 among the different standard deviations. In another experiment, sets of Gaussian kernels with standard deviations of {σ₁}, {σ₁, σ₂}, {σ₁, σ₂, σ₃}, {σ₁, σ₂, σ₃, σ₄}, {σ₁, σ₂, σ₃, σ₄, σ₅}, and {σ₁, σ₂, σ₃, σ₄, σ₅, σ₆} are used to evaluate the effect of the number of kernels (i.e., k) on PSNR. The results are presented in FIG. 7(d). As shown, the number of Gaussian kernels change the PSNR but, similar to standard deviation of the kernels, the changes are less than 0.2 among the different numbers of kernels. In term of computation time, the use of k=6 kernels doubles the computation time in comparison with that of k=2. Note that the computation time depends on both the number of iterations and the duration of each iteration. Thus, a more complex and time consuming calculation of the weights does not necessarily result in a slower algorithm as fewer iterations may be required to reach a solution.

In another experiment, to evaluate the effect of number of iterations and number of times of weight updates on PSNR, the threshold ε is changed and step 7 of the example algorithm is repeated twice, resulting in three weight updates. The PSNR curves with two weight updates are presented in FIG. 7(e) (PSNR with the third weight update was not shown for better visualization). The PSNR slightly decreased as ε increases; the computation time increases with the additional weight update. However, the third weight update did not notably change the PSNR (less than 0.08 from that with two weight updates) and actually slightly decreases PSNR for ε=0.05 and ε=0.03. Thus two weight updates are sufficient to achieve reasonable PSNR.

Lastly, FIG. 7(f) shows the effects of slice thicknesses on PSNR. As expected, PSNR decreases with increased slice thickness.

The method disclosed herein can also be used to upsample ROIs drawn on LR images. The ROIs may be manually drawn in a HR image if a reliable automated tool to outline the regions of interest is not available. The method disclosed herein can be used to upsample ROIs using an HR image with a similar contrast. The algorithm to upsample ROIs is similar to the one described above with two differences: 1) partial volume condition—i.e., Eqn. (11)—is not used because the values in the RIOs are all constant; 2) the weights are calculated only once using the HR image (i.e., step 7 of the algorithm is skipped).

The method disclosed herein can also be used to coregister LR images. The LR images may be upsampled by a HR image separately and then coregistered to each other. This may be applied for accurate correction of motion artifacts in dynamic MRI scans.

In upsampling dynamic MRI in which a contrast agent is injected, HR images before (pre-contrast) and after (post-contrast) acquisition of dynamic MRI may be acquired and used to upsample each time point (frame) of the dynamic MRI. The weights for upsampling each frame of dynamic MRI may be calculated by combining the weights from pre-contrast and post-contrast HR MRI. In one configuration, the weights for each frame are a linear combination of weights of pre-contrast and post-contrast HR images where the coefficients of weights are calculated based on similarity of that frame to each of pre-contrast and post-contrast HR MRI. This makes the method significantly faster as updating the weights (step 7 of the algorithm) will be significantly faster.

The method disclosed herein can also used to reconstruct images undersampled in the k-space in compressed sensing. In this technique, an undersampled image of a first contrast is reconstructed with the help of a HR image of a second contrast. Similar method is used here with the difference that the LR is replaced by the undersampled image and the constraint in equation (11) is replaced by a constraint in the k-space.

The method disclosed herein can also used on the baseline image of LR dynamic MRI scans. The method may also be applied to each time point of the dynamic image but the parameters therein should be adjusted to minimize the total reconstruction time so not to affect the dynamic imaging.

The method disclosed herein is not limited to the LR and HR images having different contrasts (e.g., LR T_{2W} and HR T_{1W} images). Indeed, if the LR and HR images have similar contrasts, the method disclosed herein result in higher PSNR due to their contrast similarity than the LR and HR images having different contrasts. This may be applied in upsampling dynamic images because a static HR image has similar contrast.

The method disclosed herein work robustly when image resolution or contrast changes. The method works successfully on the LR images, both with anisotropic and isotropic voxel sizes. Other feature vectors may be used estimate the similarity of the voxels on a LR image with laminar structures. But feature vectors comprising of image intensity, gradient, and multi-resolution Gaussian filtering are simple and produce reasonably accurate results in a reasonable time. The assumption of laminar structures is valid in healthy brain tissues and abnormal tissues. The method disclosed herein yields superior results in healthy and abnormal tissues, compared to other methods. Noise, intensity nonuniformity and motion artifact in the HR image, and susceptibility artifact in the LR image may affect the performance of the method disclosed herein. Intensity artifacts in the LR image may remain in the upsampled image. Nevertheless, with the presence of the noise, nonuniformity, and motion and susceptibility artifact, interpolated images using the method disclosed herein have higher image quality than other methods.

Susceptibility artifact causes large geometric distortion in the LR image. As a result, the calculated weights using the HR image do not correspond to the correct voxels in the LR image. To reduce this effect, a nonrigid registration technique may be used to register the HR image to the LR image. The registration needs to be smooth enough for the HR image to avoid following the blocky edges of the LR image interpolated by the NN algorithm (step 2 of the algorithm). After upsampling, the inverse registration may be applied to correct geometric distortion of the upsampled image.

The HR image should have some structural similarity to the LR image. If multiple HR images are used, the one closest in contrast to the LR image may be chosen or the HR images may be combined. The weights may be generated by multiplying the P(v, k) values of the two HR images.

The method disclosed in the present application results in higher interpolation accuracy—particularly near edges of images—and also requires fewer computations and is significantly faster than other methods. Although the accuracy decreases as slice thickness increases, the differences of accuracy between using the method disclosed herein and other methods also increase. This means that the interpolation accuracy using the method disclosed herein does not deteriorate as much as other methods as slice thickness increases.

The present invention has been described in terms of one or more preferred embodiments, and it should be appreciated that many equivalents, alternatives, variations, and modifications, aside from those expressly stated, are possible and within the scope of the invention.

1. A method for generating magnetic resonance imaging (MRI) images of a subject, the steps of the method comprising:
a) acquiring low resolution (LR) MRI images of a first contrast and high resolution (HR) MRI images of a second contrast;
b) generating HR images of the first contrast by interpolating the LR MRI images of the first contrast to desired resolutions with a first interpolation method;
c) coregistering the HR images of the second contrast with the HR MRI images of the first contrast to generate coregistered HR images of the second contrast and coregistered HR images of the first contrast;
d) estimating first weights using laminar structures of the coregistered HR images of the second contrast;
e) generating first interpolated HR images of the first contrast using the first weights and the coregistered HR images of the first contrast;
f) estimating second weights based on the first interpolated HR images of the first contrast and the coregistered HR images of the second contrast using laminar structures of the first interpolated HR images of the first contrast and the laminar structures of the coregistered HR images of the second contrast; and
g) generating final interpolated HR images of the first contrast using the second weights and the first interpolated HR images of the first contrast

2. The method as recited in claim 1, wherein
step d) further comprising
i) detecting first edges of the coregistered HR images of the second contrast;
ii) propagating first edge information of the coregistered HR images of the second contrast by filtering the coregistered HR images of the second contrast with blurring filters and therein generating first blurred images;
iii) generating first feature vectors of the coregistered HR images of the second contrast, wherein the first feature vectors include image intensities of the coregistered HR images of the second contrast, the first edges, and the first blurred images;
iv) generating first probability for each voxel of the coregistered HR images of the second contrast that neighbors of the each voxel having the first feature vectors similar to the each voxel; and
v) generating a first weight for the each voxel based on the first probability for the each voxel;
and step (f) further comprising
vi) detecting second edges of the first interpolated HR images of the first contrast;
vii) propagating second edge information of the first interpolated HR images of the first contrast by filtering the first interpolated HR images of the first contrast with the blurring filters and therein generating second blurred images;
viii) generating second feature Vectors of the first interpolated HR images of the first contrast, wherein the second feature vectors include image intensities of the first interpolated HR images of the first contrast, the second edges, and the second blurred images;
ix) generating second probability for each voxel of the coregistered HR images of the second contrast that neighbors of the each voxel having the first feature vectors similar to the each voxel, and corresponding voxel of the first interpolated HR images of the first contrast for the each voxel that neighbors of the corresponding voxel having the second feature vectors similar to the corresponding voxel; and
x) generating a second weight for the each voxel based on the second probability for the each voxel,

3. The method as recited in claim 2, wherein
the first probability in step iv) is calculated with a first equation as P(v, k)=exp(−α_{z}∥F_{z}(v)−F_{z}(k)∥²), wherein v represents the each voxel of the coregistered HR images of the second contrast, k represents-neighbors of the each voxel, F_{z}(v) and F_{z}(k) represent feature vectors of v and k respectively, and α_{z} is a first user-chosen non-zero value;
the first weight in step v) is the first probability multiplied by
$$\frac{1}{N} ;$$
the second probability in step ix) is calculated with a second equation as P(v, k)=exp(−α_{z}∥F_{z}(v)−F_{z}(k)∥²−αₓ∥Fₓ(v)−Fₓ(k)∥²), wherein Fₓ(v) and Fₓ(k) represent feature vectors of the corresponding voxel and neighbors of the corresponding voxel respectively, and αₓ is a second user-chosen nonzero value; and
the second weight in step x) is the second probability multiplied by
$$\frac{1}{N} .$$

4. The method as recited in claim 2, wherein the blurring filters are Gaussian filters,

5. The method as recited in claim 1, wherein
the first interpolated images of the first contrast in step e) is generated by the following steps:
i) setting the HR images of the first contrast as prior images;
ii) reconstructing posterior images with a first equation x(v)≈Σ_{kεΩ(v)}w(v, k)×(k), wherein v represents each voxel of the HR images of the first contrast, x(v) represents image intensity at the each voxel, Ω(v) represents a neighborhood of the each voxel, k represents a neighbor in the neighborhood, and w(v, k) represents the first weight;
iii) generating corrected posterior images by correcting the posterior images with degradation effects;
iv) calculating differences between the prior images and the corrected posterior images;
v) setting the corrected posterior images as the prior images; and
vi) repeating step ii)-v) until the norm of the differences is less than a threshold; and
the final interpolated HR images of the first contrast in step g) are generated by the following steps:
vii) setting the first interpolated HR images of the first contrast as the prior images;
viii) reconstructing the posterior images with a second equation x(v)≈Σ_{kεΩ(v)}w(v, k)×(k), wherein v represents each voxel of the first interpolated HR images of the first contrast, x(v) represents image intensity at the each voxel, Ω(v) represents a neighborhood of the each voxel, k represents a neighbor in the neighborhood, and w(v, k) represents the second weight;
ix) generating the corrected posterior images by correcting the posterior images with degradation effects;
x) calculating the differences between the prior images and the corrected posterior images;
xi) setting the corrected posterior images as the prior images; and
xii) repeating step viii)-xi) until the norm of the differences is less than a threshold;

6. The method as recited in claim 5, where in the degradation effects include geometric transformation, partial volume, and sub-sampling.

7. The method as recited in claim 1, wherein the first interpolation method is a nearest neighbor interpolation method.

8. The method as recited in claim 1, wherein the LR MRI images of the first contrast and the HR MRI images of the second contrast acquired in step a) are of a region of interest.

9. The method as recited in claim 1, wherein sets of HR images of multiple contrasts Are acquired in step a) and HR images of a second contrast are a set among the sets that has a contrast closest to the first contrast;

10-19. (canceled)
